(19) United States
(12) Patent Application Publication
(10) Pub. No.: US 2025/0339015 A1
(43) Pub. Date: Nov. 6, 2025

(54) INTEGRATED VISUALIZATION, SUCTION, AND IRRIGATION SYSTEM FOR ENT PROCEDURES

(71) Applicants: Northwestern University, Evanston, IL (US); The Curators of the University of Missouri, Columbia, MO (US)

(72) Inventors: Claus-Peter Richter, Skokie, IL (US); Joaquin Cury, Chicago, IL (US); Xiaodong Tan, Wilmetter, IL (US); Arnaldo Rivera, Columbia, MO (US); Stephen Philip Becker, Chicago, IL (US)

(21) Appl. No.: 19/187,717

(22) Filed: Apr. 23, 2025

Related U.S. Application Data
(60) Provisional application No. 63/642,237, filed on May 3, 2024.

(51) Int. Cl. A61B 1/00; A61B 1/015; A61B 1/018; A61B 1/06; B33Y 10/00; B33Y 70/00; B33Y 80/00

(52) U.S. Cl. CPC A61B 1/0011; A61B 1/015; A61B 1/018; A61B 1/0661; B33Y 70/00; B33Y 80/00; B33Y 10/00

(57) ABSTRACT

A method of forming an endoscopic device includes forming, by a three-dimensional (3D) printer, a first tube of an endoscopic sleeve, where the first tube is sized to receive an imaging device. The method also includes forming, by the 3D printer, a second tube of the endoscopic sleeve such that at least a portion of the second tube is mounted to the first tube, where the second tube is sized to connect to an irrigation system. The method further includes forming, by the 3D printer, a third tube of the endoscopic sleeve such that at least a portion of the third tube is mounted to one or more of the first tube and the second tube, where the third tube is sized to connect to a vacuum system.

### CROSS-REFERENCE TO RELATED APPLICATION

The present application claims the priority benefit of U.S. Provisional Patent App. No. 63/642,237 filed on May 3, 2024, the entire disclosure of which is incorporated by reference herein.

### REFERENCE TO GOVERNMENT RIGHTS

This invention was made with government support under grant number DC018666 awarded by the National Institutes of Health. The government has certain rights in the invention.

### BACKGROUND

Otolaryngology refers to surgery performed on a patient's head or neck to correct or treat a problem with the patient's ears, nose, or throat. Such procedures are also referred to as ear, nose, and throat (ENT) procedures. ENT procedures are generally conducted in very small spaces that require precise instrument placement and maneuvering. As a result, many ENT procedures utilize visualization through an endoscope or other imaging device to assist with the surgery. This endoscope is placed into the surgical area such that the surgeon can visualize the area. Depending on the procedure, the surgeon may also need to insert an irrigation tube into the surgical area to wash the area, clean the endoscope camera lens, etc. The surgeon may also utilize a vacuum (or suction) tube to remove fluid, blood, and other debris from the surgical site.

### SUMMARY

A method of forming an endoscopic device includes forming, by a three-dimensional (3D) printer, a first tube of an endoscopic sleeve, where the first tube is sized to receive an imaging device. The method also includes forming, by the 3D printer, a second tube of the endoscopic sleeve such that at least a portion of the second tube is mounted to the first tube, where the second tube is sized to connect to an irrigation system. The method further includes forming, by the 3D printer, a third tube of the endoscopic sleeve such that at least a portion of the third tube is mounted to one or more of the first tube and the second tube, where the third tube is sized to connect to a vacuum system.

The method can also include forming the endoscopic sleeve at an orientation such that the first tube of the endoscopic sleeve is perpendicular to a ground surface upon which the 3D printer is positioned. In one embodiment, the endoscopic sleeve is formed from MED-AMB 10 material, MED-WHT 10 material, or PRO-BLK 10 material. In another embodiment, forming the second tube comprises forming an end portion of the second tube, where the end portion of the second tube is formed at a non-zero degree angle relative to the first tube such that the end portion of the second tube is not in direct contact with the first tube. The method can also include forming at least a portion of the end portion of the second tube as an irrigation system connector.

In another embodiment, forming the third tube comprises forming an end portion of the third tube, where the end portion of the third tube is formed at an angle relative to the first tube such that the end portion of the third tube is not in direct contact with the first tube. In one embodiment, the end portion of the third tube is parallel to the end portion of the second tube. The method can also include forming at least a portion of the end portion of the third tube as a vacuum system connector.

The method can further include forming, by the 3D printer, a structural support for the endoscopic sleeve, where the structural support includes a first portion that extends between the first tube and the end portion of the second tube. In one embodiment, the first portion of the structural support is formed to be perpendicular to the first tube. In another embodiment, the structural support includes a second portion that extends between the end portion of the second tube and the end portion of the third tube. In one embodiment, the second portion of the structural support is formed to be parallel to the first tube. In another embodiment, the first tube is sized to receive a light source to provide illumination for the imaging device. The method can also include forming a distal end of the second tube such that the distal end is angled toward a distal end of the first tube such that the irrigation system is able to clean a lens of the imaging device. The method can also include cleaning an interior and an exterior of the endoscopic sleeve with alcohol.

An illustrative endoscopic system includes a first tube of an endoscopic sleeve, where the first tube is sized to receive an imaging device. The system also includes a second tube of the endoscopic sleeve, where the second tube is sized to connect to an irrigation system. The system also includes a third tube of the endoscopic sleeve, where the third tube is sized to connect to a vacuum system. The system further includes a controller in communication with the vacuum system and the irrigation system such that the controller controls the vacuum system and the irrigation system.

In one embodiment, the second tube includes an end portion that is formed at a non-zero degree angle relative to the first tube such that the end portion of the second tube is not in direct contact with the first tube. In another embodiment, the third tube includes an end portion that is formed at an angle relative to the first tube such that the end portion of the third tube is not in direct contact with the first tube and such that the end portion of the third tube is parallel to the end portion of the second tube. In another embodiment, the system includes a structural support for the endoscopic sleeve, where the structural support includes a first portion that extends between the first tube and the end portion of the second tube. The system can also include a second portion of the structural support that extends between the end portion of the second tube and the end portion of the third tube.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the invention will hereafter be described with reference to the accompanying drawings, wherein like numerals denote like elements.

FIG. 1 is a schematic representation of the integrated system for performing ENT procedures in accordance with an illustrative embodiment.

FIG. 2A is a side view of a probe for performing visualization, suction, and irrigation during an ENT procedure in accordance with an illustrative embodiment.

FIG. 2B is a partial view showing a distal end of the probe in accordance with an illustrative embodiment.

FIG. 3 shows a prototype of a probe device printed in a resin material in accordance with an illustrative embodiment.

FIG. 4A depicts an example valve to control suction or irrigation in accordance with an illustrative embodiment.

FIG. 4B depicts dual foot pedals for controlling the suction and irrigation valves in accordance with an illustrative embodiment.

FIG. 5 is a table that depicts the main mechanical properties of various biocompatible materials that can be used to form the proposed system in accordance with an illustrative embodiment.

FIG. 6 depicts failure of a 3D printed sleeve manufactured using a biocompatible material with lower tensile strength (ultimate) (29.1 M Pa) and tensile modulus (994 M Pa) than materials in the table of FIG. 5 in accordance with an illustrative embodiment.

FIG. 7A shows a 3D printed sleeve design using a different biocompatible material (BioMed Durable) from those in the table of FIG. 5, showing the higher density of support structure required to hold the piece while printing in accordance with an illustrative embodiment.

FIG. 7B shows the same sleeve design using the material PRO-BLK 10 along with the minimal support utilized to support the sleeve during production in accordance with an illustrative embodiment.

FIG. 8A depicts an endoscopic sleeve that is printed at a horizontal angle of 60° in accordance with an illustrative embodiment.

FIG. 8B depicts an endoscopic sleeve that is printed vertically (i.e., at an angle of 90° relative to the ground/floor surface) in accordance with an illustrative embodiment.

FIG. 9 depicts that positioning the endoscopic sleeves in a vertical orientation allows for a higher number of sleeves to be printed simultaneously, improving production throughput in accordance with an illustrative embodiment.

FIG. 10 depicts the endoscopic sleeve with a structural support (L-shape) for the irrigation and suction ports to help strengthen the device during use in accordance with an illustrative embodiment.

FIG. 11 depicts an endoscopic sleeve manufactured with the material PRO-BLK 10 (3D Systems), and with the inlet ports for irrigation (I) and suction(S) connected via a structural support (L-shape) to strengthen the device during its use and avoid fractures in accordance with an illustrative embodiment.

FIG. 12 depicts a computing device in direct or indirect communication with a network in accordance with an illustrative embodiment.

### DETAILED DESCRIPTION

In the last decades, endoscopic technology has improved surgery and exploratory interventions, enabling precise diagnosis and treatment of areas that were previously difficult to access without significant tissue disruption. This advancement has notably influenced the field of ear, nose, and throat (ENT) medicine, where its use has expanded the range of procedures achievable with greater accuracy and shorter recovery periods. Nevertheless, current endoscopic devices are not without limitations. One major challenge is the separation of the endoscopic system from irrigation and suction equipment, which are essential for maintaining cleanliness and visibility. This separation requires clinicians to operate multiple devices simultaneously, complicating navigation and increasing the procedural complexity within the limited space of a surgical/exploratory area. Furthermore, the flexibility of current endoscopes is limited by their bending radius. The reliance on optical fibers for illumination often results in reduced light propagation efficiency when navigating tight bends, restricting the visualization of hard-to-reach locations. Similarly, devices that use fiber bundles for image acquisition encounter similar constraints, affecting the quality of the images obtained. Additionally, the use of articulated manipulators in tight surgical fields raises the risk of inadvertently damaging surrounding tissues, underscoring the need for improvements in endoscopic design and functionality to enhance the efficacy and safety of ENT procedures.

In addition to the above-discussed constraints, current endoscopic, irrigation and suction devices are crafted from medical-grade metallic alloys, which contribute to their weight. This becomes an issue during surgeries that require the surgeon to handle these devices for extended periods, leading to discomfort and potential strain. Such concerns are relevant in both clinical and veterinary practices. Moreover, the non-transparent surface of these suction devices introduces another challenge. When a blockage occurs, identifying the precise location of the clog is difficult. This opacity complicates the process of quickly addressing and resolving obstructions.

Described herein is a system that includes an imaging device (e.g., chip-camera) integrated in a novel fashion with a suction and irrigation system designed for ear, nose, and throat (ENT) surgery and exploratory interventions. In one embodiment, the device includes two distinct component blocks. The first block includes an endoscopic probe containing three tubing profiles, which can be 3D printed in one embodiment. One tubing profile accommodates a camera/endoscope and an illumination source, the second tubing profile can be dedicated to irrigation, ensuring the surgical or exploration field is clean along with the camera/endoscope lens. The third tubing profile can be purposed for suction, efficiently removing fluids and debris. These profiles can be either rigid or flexible (e.g., based on the 3D printing material or other material chosen), allowing them to adapt to different ENT needs. The combination of the camera's size and the tubing's flexibility enhances the ability to access tight and hard-to-reach areas, offering a benefit across a wide range of procedures, such as, tympanoplasty, cholesteatoma removal, mastoidectomy, stapedectomy, cochlear implant surgery, sinus treatments, nasal surgeries, adenoidectomy, and laryngoscopy for throat conditions, amongst other clinical applications. The second block of the system can include electronic drivers for the visualization system and two valves (one for suction and one for irrigation) that can be controlled via pedals, switches, etc. In an illustrative embodiment, a video signal from the camera/endoscope can be sent to a computer/screen via HDMI/USB such that a system operator can visual the procedure as it is being performed.

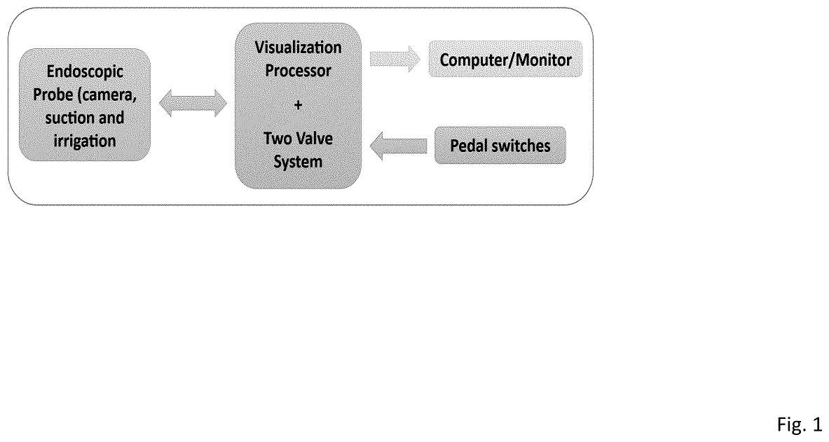

FIG. 1 is a schematic representation of the integrated system for performing ENT procedures in accordance with an illustrative embodiment. As discussed above, the system has two main blocks. The first block is an endoscopic probe containing a camera tube, a suction tube, and an irrigation tube. The second block includes a computer processor for performing video signal analysis and post-processing. Any type of computer processor and/or computing system may be used to perform the analysis and processing. The system also includes two valves, a first valve to control suction and a second valve to control irrigation. In an illustrative embodiment, the valves are controlled via foot pedal switches that regulate the irrigation and suction rates. Alternatively, hand-controlled pedals, switches, or any other controller may be used to operate the valves. The video signal captured by a camera positioned in the camera tube is sent to a computer monitor via a universal service bus (USB) connection, a high-definition multimedia interface (HDMI) connection, a wireless connection, etc.

Most currently available endoscopic devices, irrigation systems, and suction systems are made of metallic alloys which is problematic in terms of weight when the surgeon performs time consuming surgical procedures. Conversely, the proposed device can be 3D printed or otherwise formed from lightweight biocompatible plastic material. Specific examples of plastic materials are included below. Alternatively, a different biocompatible material may be used such as silicon or rubber. The use of such materials results in a system that not only integrates the three functions discussed above (visualization, irrigation and suction), but that is also easy to handle and straightforward to manufacture it (not only from an engineering point of view but also in cost of production).

Additionally, unlike traditional devices, the proposed system includes an irrigation tubing for cleaning the surgical field. Inclusion of irrigation not only alleviates the use of multiple separate devices by the surgeon, but also provides a way to clean the camera/endoscopic lens in situ. This is extremely important to improve surgical efficiency and limit patient discomfort. In traditional systems, when the endoscope lens becomes dirty and proper visualization is compromised, such as during rhinology surgeries, the surgeon must remove the endoscope from the patient's body to clean the lens before reinserting it to continue the procedure. This process happens multiple times in a surgery, becoming a tedious and time consuming process. Furthermore, depending on the complexity of the surgery, placing the endoscope back in the same position multiple times can be challenging.

In another illustrative embodiment, the system is designed to be biocompatible, clear/transparent (important in at least the suction tube so that any clogged region can be easily identified), and with the capability of being sterilized (thermal, chemical, radiation) such that the probe can be reused. To this end, in one embodiment, the probe proposed is 3D printed in a biocompatible material that has high transparency and mechanical resistance. Alternatively, a different type of biocompatible material may be used to form the probe.

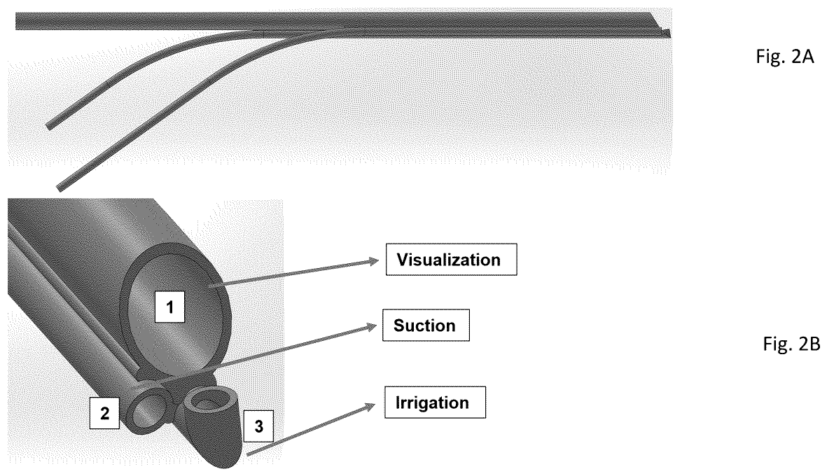

One embodiment of the probe that was designed using the software Solidworks is shown in FIG. 2. Specifically, FIG. 2A is a side view of a probe for performing visualization, suction, and irrigation during an ENT procedure in accordance with an illustrative embodiment. FIG. 2B is a partial view showing a distal end of the probe in accordance with an illustrative embodiment. In the longitudinal view of FIG. 2A, there can be seen three tubing profiles. One tubing profile is for the visualization system, and the remaining two are for suction and irrigation. In the case of the irrigation tube, as shown in FIG. 2B, a distal end of the tube has a bending angle towards the visualization tube. The bending angle can be a non-zero degree angle, such as 20 degrees, 45 degrees, 60 degrees, 90 degrees, etc. depending on the implementation. This is done so that the irrigation solution used for washing the surgical field can also employed to clean the camera lens. The length of the tubing varies from 100 to 175 millimeters (mm) for different needs. In one embodiment, the size of each tube can be as follows. The visualization tube 1 can have an inner diameter can range from 1 to 4 mm and an outer diameter from 1.5 to 4.5 mm. The irrigation and suction tubes can have an inner diameter of 1.5 mm and an outer diameter 2.1 mm. In alternative embodiments, different ranges or values may be used.

The reason for having a range in the inner and outer diameter for the visualization tube is due to two possible scenarios. The former is that in some hospitals, there are conventional endoscopes (e.g., rigid endoscopes) ready to be used. Such a conventional endoscopic system will have a diameter of ˜3.9 mm in diameter, and the irrigation tube can be sized to receive such conventional systems, along with the irrigation and suction systems that can be easily integrated. The smaller inner and outer diameters proposed for the visualization tube are to accommodate a miniaturized camera (e.g., the smallest camera in the world and similar devices) and light sources (e.g., light-emitting diodes (LEDs)), such that the integrated system can be employed in applications (clinic and veterinary) where it is necessary to use small devices to access hard-to-reach areas. Furthermore, in FIG. 2, it can be seen that suction and irrigation tubes 2 and 3 have a bending radius in their distal part. This bending is intended to facilitate a better handling for the surgeon. In one embodiment, the distal portions of the suction and irrigation tubes can include universal connectors that are used to connect to separate irrigation and suction devices so any clinic can use them.

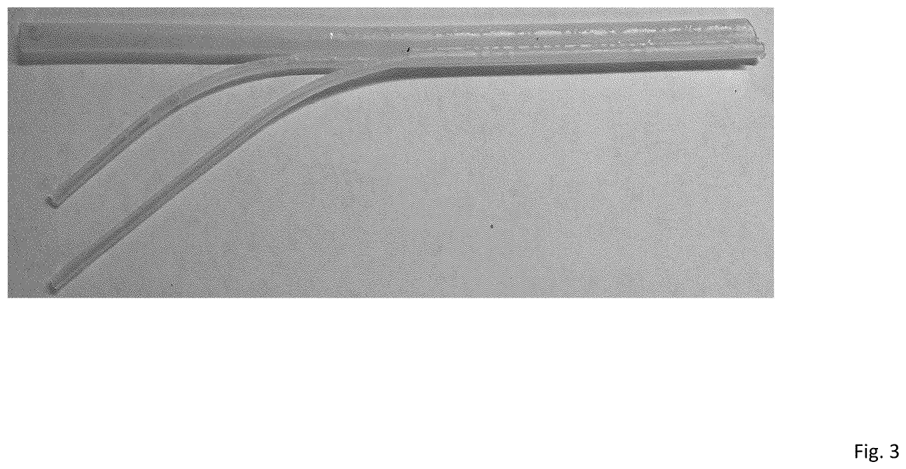

FIG. 3 shows a prototype of a probe device printed in a resin material in accordance with an illustrative embodiment. As mentioned previously, this design can be used for conventional endoscopes (diameter 4 mm) where no irrigation and suction systems are available. This device can also be used for applications where the area is small, requiring smaller optical systems. In this scenario, the system can use a miniaturized camera (e.g.,—OVM 6948—from Omnivision, USA and similarly sized cameras). Such a miniature camera can be less than 2.5 mm in width/thickness and can have several pixels of resolution and dimensions, depending on the surgeon's needs.

In an illustrative embodiment, the camera can be removably mounted to a distal end of the visualization tubing. Using such an approach, in the event that the miniaturized camera is not working appropriately due a manufacturing issue, instead of removing the entire probe from the surgical area, the camera can be easily removed from the visualization tube and replaced with a new camera without affecting the positioning of the 3 tubing profiles of the probe. To delivery light to the surgical/exploration area, micro-LEDs (size 0.65×0.35×0.2 mm) or flexibles waveguides (100 μm diameter) can be employed. Alternatively, a different size and/or type of light source may be used.

Referring again to FIG. 2A, the second block of the system includes the visualization drivers/processors so the video signal from the camera can be processed and transmitted to a computer monitor (or memory) via USB, HDMI, or other connection. The images and video acquired by the miniaturized camera are analyzed with a post-processing module, which can be part of any type of computing system. The module can include two main components: a video bridge chip and a digital signal processor (DSP). The former converts the camera's analog video signals into a digital format. After this initial processing, the DSP further processes the digital signals.

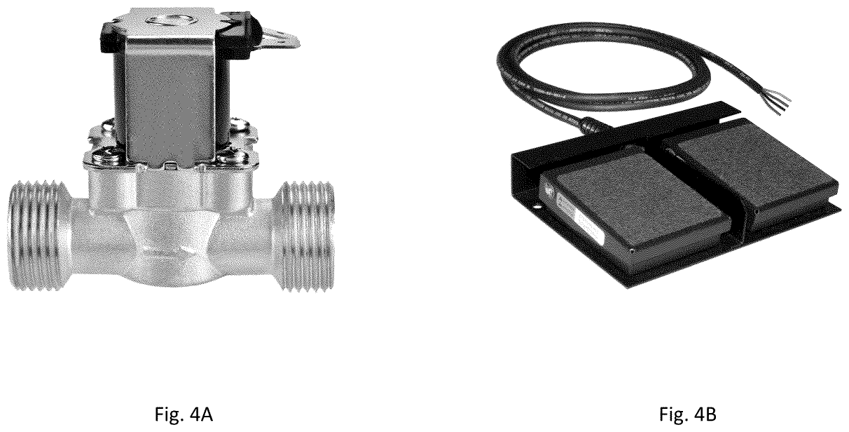

In addition to the visualization processor, the system includes two electrical valves, one for irrigation and one for suction, that can be controlled (open and close states) externally using a dual foot pedal switch or other controller. In an illustrative embodiment, a box containing the valves will have two connections, one to the irrigation source and one to the vacuum source. These connectors can be universal so they can be used in any clinic, hospital, etc. FIG. 4A depicts an example valve to control suction or irrigation in accordance with an illustrative embodiment. FIG. 4B depicts dual foot pedals for controlling the suction and irrigation valves in accordance with an illustrative embodiment. In alternative embodiments, different components may be used to control the irrigation and suction systems.

As discussed above, in one embodiment the proposed system can be formed through additive manufacturing (i.e., 3D printing). Included below is a description of materials used and illustrative operations in the manufacturing process. In an illustrative embodiment, the sleeve is printed using a biocompatible material with suitable mechanical properties and the ability to withstand conventional clinical sterilization. MED-AMB 10 (3D Systems) is a material that fulfills these requirements, offering rigidity, moisture resistance, thermal stability above 100° C., chemical compatibility and isotropic properties for consistent performance in all directions. The material MED-AMB 10 meets ISO 10993-5 and -10 standards for biocompatibility (cytotoxicity, sensitization, and irritation), making it appropriate for medical applications such as surgical guides, fluid handling components, and parts requiring high-definition detail. The material's transparency allows for easy detection of debris or clogs that may obstruct the suction channel, helping maintain proper irrigation and visualization during use. Additionally, the MED-AMB 10 material is sterilizable by autoclave, ensuring compatibility with standard clinical protocols.

An alternative material that can be used to form the proposed system is MED-WHT 10 (3D Systems). MED-WHT 10 is a rigid, white material suitable for medical applications involving biocompatibility, sterilization, or thermal resistance. The material meets ISO 10993-5 and -10 standards (covering cytotoxicity, sensitization, and irritation) and is autoclavable, with thermal resistance above 100° C. and excellent humidity resistance. While it delivers high precision and fine feature detail comparable to MED-AMB 10, MED-WHT 10 has a higher tensile modulus (making it stiffer) but a slightly lower tensile strength. In practical terms, a stiffer material will bend less under moderate loads but may fail under a lower maximum load, whereas a material with higher tensile strength can handle greater force overall before breaking but may flex more. Additionally, MED-WHT 10 has a lower elongation at break (3%) compared to MED-AMB 10 (4%), making it more brittle and less tolerant to deformation before failure.

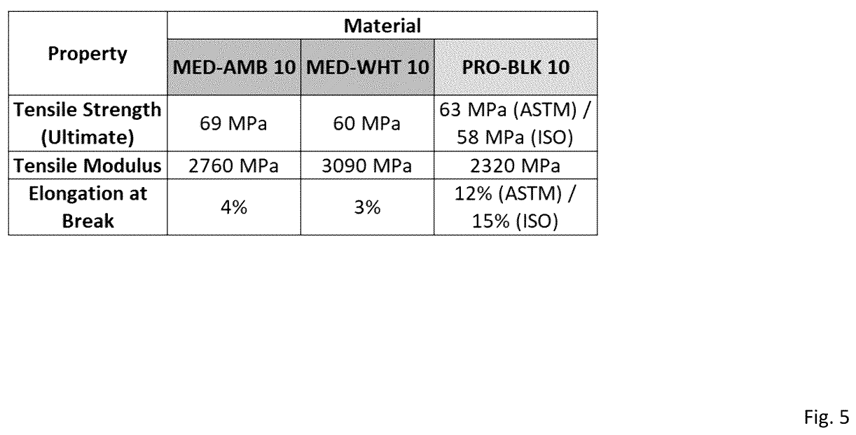

Another alternative material that can be used to form the proposed system is PRO-BLK 10 (3D Systems). PRO-BLK 10 is a black biocompatible material option that meets ISO 10993 standards, making it suitable for medical use. It offers lower stiffness than both MED-AMB 10 and MED-WHT 10, as reflected by its lower tensile modulus, but it exhibits comparable tensile strength and significantly higher elongation at break (12-15%), indicating greater flexibility and capacity to stretch before failure. This combination makes PRO-BLK 10 more resistant to cracking under stress, although it may not provide the same rigidity as the other two materials. FIG. 5 is a table that depicts the main mechanical properties of various biocompatible materials that can be used to form the proposed system in accordance with an illustrative embodiment. In alternative embodiments, different biocompatible material(s) may be used.

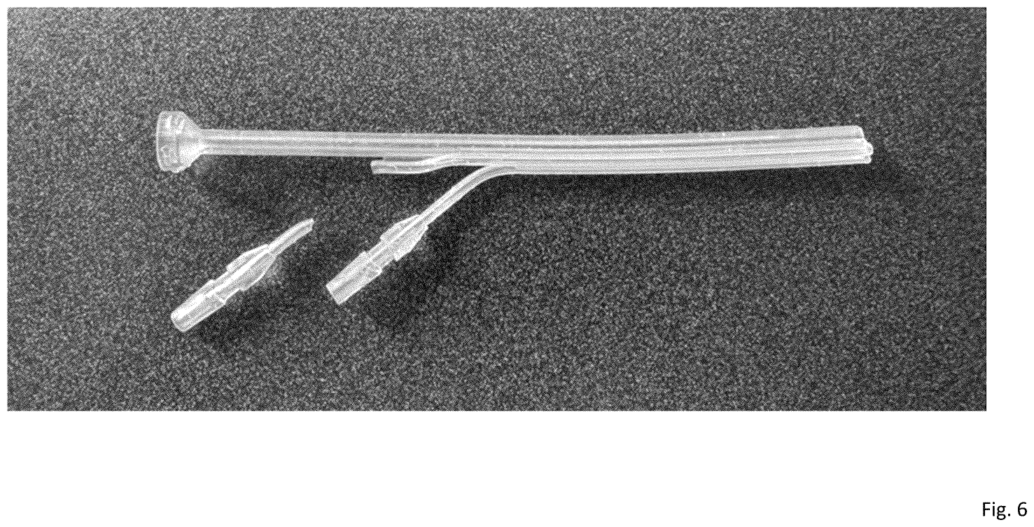

Choosing an inappropriate material for making the 3D-printed sleeve can lead to several issues that compromise device performance. If the mechanical properties—such as tensile strength, stiffness, and elongation at break—are insufficient for the intended application, the part may crack or break under normal use. For example, FIG. 6 depicts failure of a 3D printed sleeve manufactured using a biocompatible material with lower tensile strength (ultimate) (29.1 M Pa) and tensile modulus (994 M Pa) than materials in the table of FIG. 5 in accordance with an illustrative embodiment. As shown, the device of FIG. 6 is fractured and bent, illustrating the importance of selecting materials with appropriate tensile strength and tensile modulus.

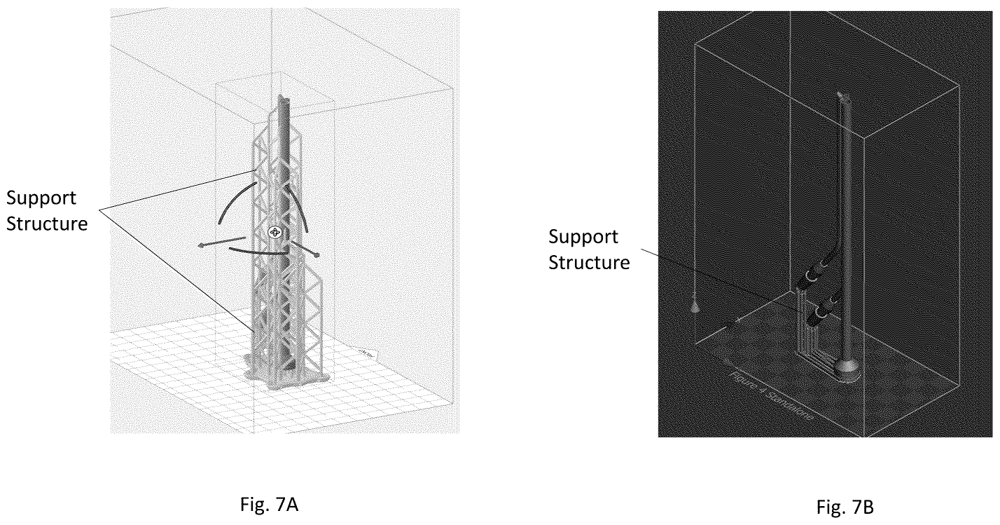

Even if a material meets biocompatibility standards, it must also maintain stability under clinical sterilization methods. Otherwise, repeated autoclaving or exposure to high temperatures can degrade the device. In addition, some materials demand excessive support structures during printing, which not only increases material usage and production time but also leaves more surface artifacts that require extensive postprocessing. These factors collectively can result in higher costs, delayed clinical use, and diminished reliability of the final medical device. FIG. 7A shows a 3D printed sleeve design using a different biocompatible material (BioMed Durable) from those in the table of FIG. 5, showing the higher density of support structure required to hold the piece while printing in accordance with an illustrative embodiment. FIG. 7B shows the same sleeve design using the material PRO-BLK 10 along with the minimal support utilized to support the sleeve during production in accordance with an illustrative embodiment. As shown, the support utilized to facilitate printing in FIG. 7B is considerably less dense than that required by the material of FIG. 7A, leading to less printing material and marks.

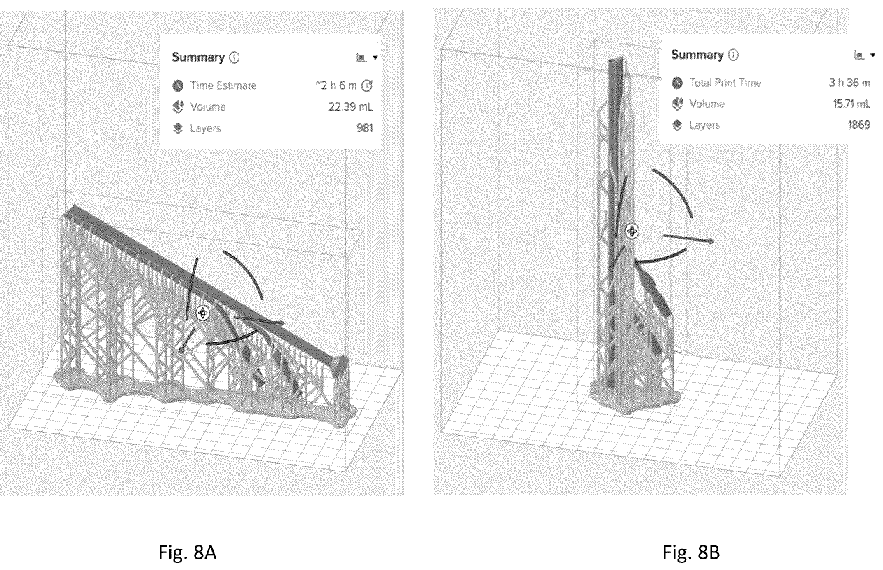

Positioning of the sleeve/system in a 3D printer is discussed below. To preserve geometry and minimize support structures on critical surfaces, the sleeve should be printed in a vertical (90°) orientation relative to a flat ground/base surface upon which the 3D printer is positioned. Printing at an angle different from 90° may reduce overall build time but typically requires more support material, which increases the likelihood of surface marks and necessitates further postprocessing. FIG. 8A depicts an endoscopic sleeve that is printed at a horizontal angle of 60° in accordance with an illustrative embodiment. FIG. 8B depicts an endoscopic sleeve that is printed vertically (i.e., at an angle of 90° relative to the ground/floor surface) in accordance with an illustrative embodiment. As shown, the vertically printed endoscopic sleeve of FIG. 8B requires significantly less support material as compared to the endoscopic sleeve printed at the horizontal angle of 60° in FIG. 8A. Also, although the overall printing time is reduced in the orientation of FIG. 8A, the endoscopic sleeve of FIG. 8A requires extensive post-processing to remove and polish the support marks along the sleeve surface. As a result, the overall production time is lower for the endoscopic sleeve printed vertically as shown in FIG. 8B.

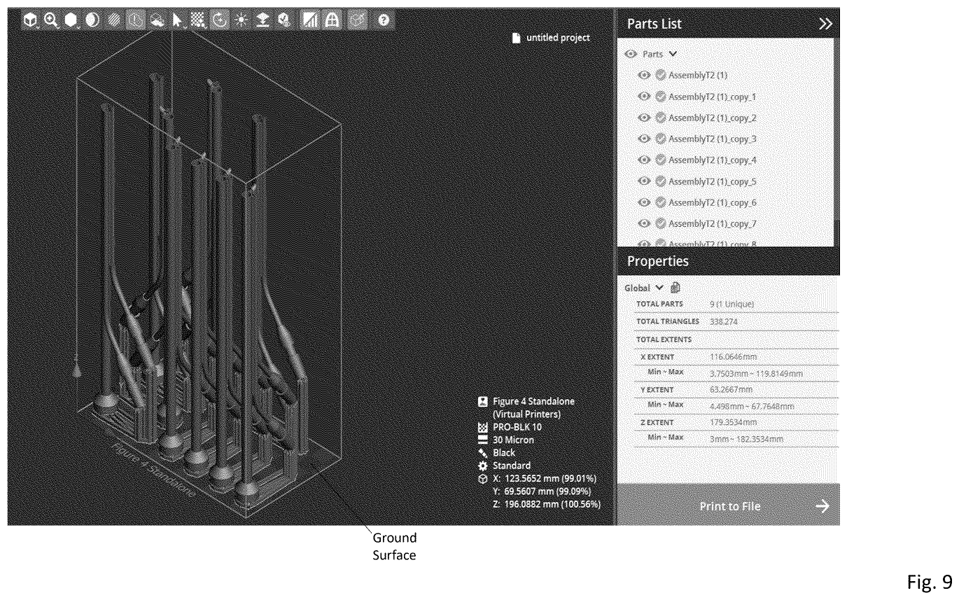

Additionally, the inventors found that non-vertical print orientations can introduce anisotropy in layer adhesion, leading to greater susceptibility to bending and structural weaknesses under mechanical stress, which could result in device failure. Printing vertically also allows for more efficient use of the build platform, enabling a higher number of sleeve designs to be printed simultaneously, which improves production throughput. FIG. 9 depicts that positioning the endoscopic sleeves in a vertical orientation allows for a higher number of sleeves to be printed simultaneously, improving production throughput in accordance with an illustrative embodiment.

The inventors also found that orienting the endoscopic sleeve upside down (i.e., with the proximal end down) ensures that the tilted tip of the irrigation tube is printed last. As used herein, the distal end of the endoscopic sleeve (or probe) is the end which first enters the patient and the proximal end of the endoscopic sleeve is the opposite end, which includes connections to the vacuum/irrigation systems. The close up in FIG. 2B shows the distal end of the sleeve. The upside down printing orientation also allows uncured material to flow away from the sleeve during printing, minimizing the risk that residual material becomes cured in unintended areas, which could affect the geometry and proper functioning of the sleeve. In addition, this configuration in the printer allows the reduction of support material while producing since the irrigation and suction inlets ports are closer to the platform where the printed design is fixed.

If the endoscopic sleeve is printed at an angle other than vertical, structural deformation may occur, resulting in bent or warped features. Printing the sleeve in an upright (not upside-down) position allows uncured material to accumulate in the irrigation tip and cure within the sleeve, potentially blocking the irrigation tube. Moreover, any angled orientation increases the need for support structures, leading to higher material usage, increased printing costs, and extended post-processing time. Even with careful post-processing, support marks may remain on the sleeve's tubing section, creating weak points that could result in cracks or mechanical failure during use.

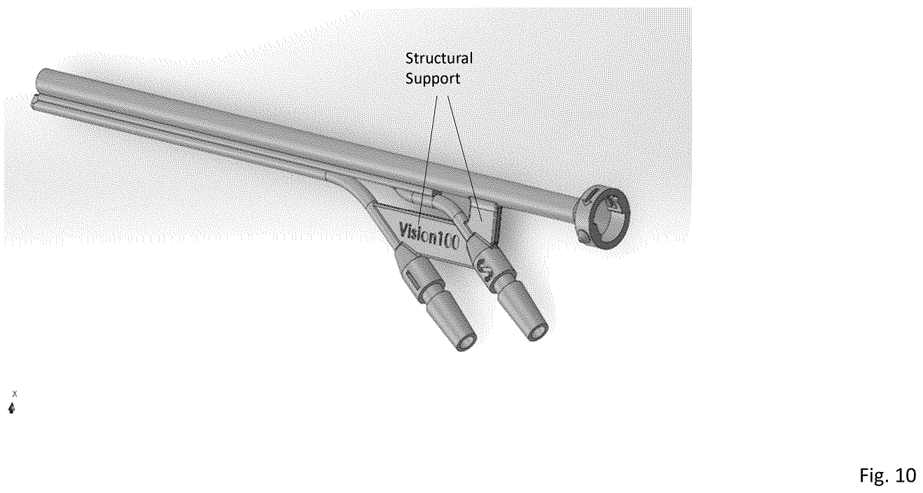

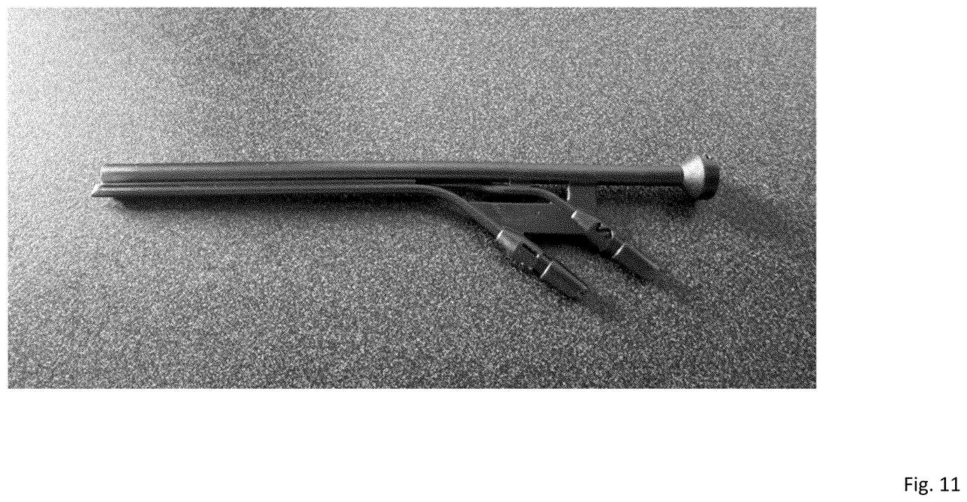

To improve structural stability of the endoscopic sleeve, a reinforcement feature was added around the irrigation and suction inlet ports. This addition is intended to prevent mechanical failure in cases where clinical tubing exerts pulling forces directed away from the device's main axis-forces that could otherwise cause the inlets to crack or detach, as shown in FIG. 6. The added support enhances the strength of the device at these critical junctions, helping ensure reliable performance during clinical use. FIG. 10 depicts the endoscopic sleeve with a structural support (L-shape) for the irrigation and suction ports to help strengthen the device during use in accordance with an illustrative embodiment. FIG. 11 depicts an endoscopic sleeve manufactured with the material PRO-BLK 10 (3D Systems), and with the inlet ports for irrigation (I) and suction(S) connected via a structural support (L-shape) to strengthen the device during its use and avoid fractures in accordance with an illustrative embodiment. As shown in FIGS. 10 and 11, the structural support includes a first portion that extends from a main tube of the endoscopic sleeve to a first inlet port (e.g., a suction inlet port) and a second portion that extends between the first inlet port and a second inlet port (e.g., an irrigation inlet port). As also shown, the first portion of the structural support is perpendicular to the main tube of the endoscopic sleeve and the second portion of the structural support is parallel to the main tube of the endoscopic sleeve.

As also shown, the irrigation tube and vacuum/suction tubes each have end portions that are angled relative to the main tube (for the imaging device and/or light source(s)). The angle can be any non-zero angle such that the end portions are not in contact with the main tube. In an illustrative embodiment, the end portions of the irrigation/vacuum tubes are parallel to one another, as shown in FIG. 10. Alternatively, the end portions may not be parallel to one another. As also shown, an irrigation system connector is formed on the end portion of the irrigation tube and a vacuum system connector is formed on the portion of the vacuum tube.

After the sleeve is printed, it is important to thoroughly clean any uncured resin remaining inside the visualization, suction, and irrigation channels. To achieve this, the inventors used a print washing system that circulates isopropyl alcohol both internally and externally through the part for several minutes. Based on experience printing devices across multiple projects, it was found that when internal profiles are particularly narrow or small, the standard washing cycle alone is insufficient. In these cases, a pre- and post-wash step is used, where isopropyl alcohol is manually pumped through the narrow channels in both steps. This additional manual flushing significantly improves the effectiveness and efficiency of the automated print wash, ensuring that resin is fully removed from critical internal structures.

If the washing process is not performed correctly as mentioned, residual uncured resin may remain inside the visualization, suction, or irrigation channels. During the subsequent UV curing stage—when the resin fully solidifies—this leftover material can harden and cause permanent blockages within the channels. Once cured, these obstructions cannot be removed or modified, rendering the device non-functional and requiring a complete reprint.

As noted above, the materials selected for the device must be compatible with conventional clinical sterilization methods, such as gas sterilization and/or, autoclaving—the most widely used method in clinical practice. The resins used, such as MED-AMB 10 and MED-WHT 10, are specifically designed to withstand autoclave conditions, maintaining thermal resistance above 100° C. and moisture stability, both of which are essential to preserve mechanical integrity after sterilization. These materials are autoclavable and retain their rigidity and mechanical strength post-sterilization, ensuring the device's reliability during clinical use. Using materials with poor thermal resistance could lead to deformation or degradation during sterilization, ultimately compromising device performance. Although PRO-BLK 10 has a lower heat deflection temperature (around 70° C.), it may still be suitable for alternative sterilization methods like gas.

Transparency of the material used is another important feature for components involving fluid flow, as it allows visual inspection for blockages or clogs—particularly within the suction channel. This property is present in MED-AMB 10, whose translucent amber appearance enables easy detection of debris or obstruction during use. In contrast, the other two materials considered—MED-WHT 10 (white) and PRO-BLK 10 (black)—do not offer visual transparency due to their color. Nevertheless, despite the lack of visual inspection capability, their mechanical and thermal properties make them suitable alternatives for the endoscopic sleeve.

The curing process is a critical post-printing step that uses ultraviolet (UV) light to fully solidify the printed resin and stabilize its mechanical, thermal, and biocompatibility properties. Each material—MED-AMB 10, MED-WHT 10, and PRO-BLK 10 from 3D Systems—has specific guidelines for UV curing time, temperature, and equipment, which must be followed to achieve the validated performance of the final part. It is especially important to consider the resin's optical properties during this process. As noted in prior studies, opaque resins such as white or black formulations can limit the penetration of UV light, particularly in larger parts or those with complex internal geometries. This can result in areas of semi-cured or uncured resin, which may compromise the part's functionality, safety, or biocompatibility. Moreover, over-curing can negatively affect the mechanical performance of the material, leading to a higher fragility and reducing its strength beyond the stated specifications

Minimizing the use of support material during printing is critical for achieving a clean surface finish and simplifying the post-processing phase. Reducing support structures—especially on functional or visible surfaces—not only decreases the number of support marks but also facilitates a more efficient polishing process. Excessive support contact can lead to surface irregularities that are difficult to remove and may weaken the mechanical integrity of the device over time.

After curing and before sanding, support structures are carefully removed using a precise cutting tool to avoid introducing stress or micro-cracks at the interface. This can be done using a sharp razor blade or scalpel to ensure a clean cut with minimal force. Once the supports are removed, a multi-step sanding process can be performed, gradually refining the surface. A typical sequence includes: P150 grit for initial shaping and removal of remaining support marks, P400 grit for intermediate smoothing, and P1000 grit for final polishing and achieving a smooth, uniform surface.

Following the sanding process, a two-step isopropyl alcohol cleaning is performed—first externally and then internally—to remove any residual dust or debris from the polishing process. This ensures that no particulate matter remains within the device, especially in fluid or visualization channels.

Included below is a discussion comparing the use of biocompatible resins to various metals. The use of biocompatible resins to form the endoscopic sleeve, as discussed herein, represents a promising alternative to medical-grade metals, particularly in applications where ease of manufacturing, rapid prototyping, production scaling, and cost-efficiency are priorities. Compared to metals, resin-based 3D printing allows for faster production cycles and significantly lower production costs, making it highly suitable for the development and production of surgical tools, such as the visualization, suction and irrigation sleeve.

Medical-grade metals such as stainless steel 316L and titanium alloys are known for their superior mechanical properties, including high tensile strength, fatigue resistance, and thermal stability. These qualities make them ideal for applications requiring long-term durability and structural integrity. However, they require complex and costly fabrication processes, especially when produced using advanced 3D printing technologies such as Direct Metal Printing (DMP), which fuses metal powders with high-powered lasers. While these systems—offered by companies like 3D Systems—have continued to evolve in accuracy and resolution, the high equipment and material costs remain a barrier for many applications. Metal 3D printers are typically at least 15 times more expensive than resin-based systems, and the cost per printed part can exceed $40 without including the post-processing stage.

Biocompatible resins, on the other hand, offer a practical and scalable alternative, especially when the mechanical demands of the application do not require the full strength of metal. Resin-based 3D printing enables rapid production, high-resolution detail, and significant cost savings. The cost of manufacturing the proposed endoscopic sleeve using the selected resin is approximately $2 per unit including the post-processing stage. The resin used in the sleeve offers adequate mechanical strength, thermal resistance, and precision for its intended use in rhinology surgery and the other applications discussed herein. Furthermore, its affordability enables the practical use of single-use devices, which can help reduce the risk of cross-contamination.

Cross-contamination remains a concern in clinical settings, particularly when devices with complex internal geometries are reused and are difficult to fully sterilize. This issue has been highlighted by the FDA in cases involving reusable instruments like duodenoscopes, where retained biological material led to infection outbreaks. While the endoscopic sleeve is used in a different anatomical context, the underlying concern of ensuring that internal fluid flows remain free of residual contaminants is similar. In this context, low-cost, mechanically suitable, sterilizable, and biocompatible resins offer a safer and more practical solution for surgical tools with internal channels, supporting the development of disposable alternatives such as the endoscopic sleeve described herein.

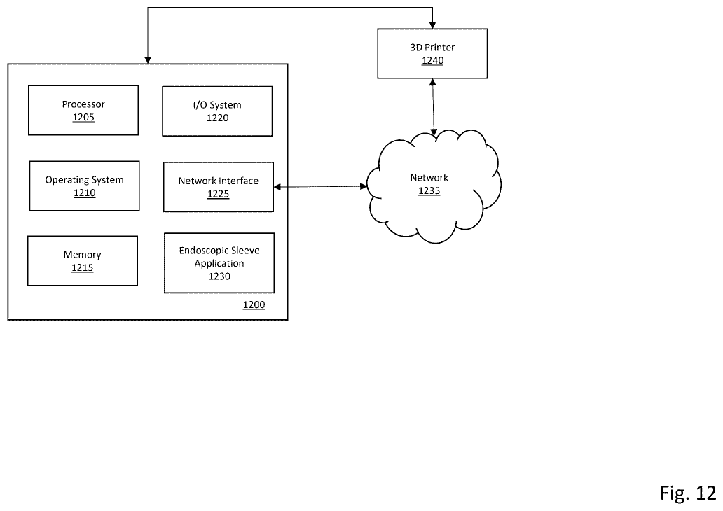

Any of the operations described herein can be performed by a computing system that includes a memory, processor, user interface, network interface, display, etc. For example, any of the operations described herein can be implemented as computer-readable instructions stored on a computer-readable medium. Upon execution of the computer-readable instructions by the processor, the computing system performs the various operations described herein to implement the system. As an example, FIG. 12 depicts a computing device 1200 in direct or indirect communication with a network 1235 in accordance with an illustrative embodiment. A 3D printer 1240 is in direct communication with the computing device 1200 and/or connected to the computing device 1200 through the network 1235. In one embodiment, the computing device can be incorporated into the 3D printer 1240. Alternatively, the computing device 1200 can be a standalone controller computer, a cell phone, tablet, laptop computer, etc.

The computing device 1200 includes a processor 1205, an operating system 1210, a memory 1215, an input/output (I/O) system 1220, a network interface 1225, and an endoscopic sleeve application 1230. In alternative embodiments, the computing device 1200 may include fewer, additional, and/or different components. The components of the computing device 1200 communicate with one another via one or more buses or any other interconnect system.

The processor 1205 of the computing device 1200 can be in electrical communication with and used to control the 3D printer 1240. The processor 1205 can be any type of computer processor known in the art, and can include a plurality of processors and/or a plurality of processing cores. The processor 1205 can include a controller, a microcontroller, an audio processor, a graphics processing unit, a hardware accelerator, a digital signal processor, etc. Additionally, the processor 1205 may be implemented as a complex instruction set computer processor, a reduced instruction set computer processor, an x86 instruction set computer processor, etc. The processor 1205 is used to run the operating system 1210, which can be standard or a custom operating system specific to the requirements of the proposed system.

The operating system 1210 is stored in the memory 1215, which is also used to store programs, endoscopic sleeve data, algorithms, network and communications data, peripheral component data, the endoscopic sleeve application 1230, and other operating instructions. The memory 1215 can be one or more memory systems that include various types of computer memory such as flash memory, random access memory (RAM), dynamic (RAM), static (RAM), a universal serial bus (USB) drive, an optical disk drive, a tape drive, an internal storage device, a non-volatile storage device, a hard disk drive (HDD), a volatile storage device, etc.

The I/O system 1220, or user interface, is the framework which enables users (and peripheral devices) to interact with the computing device 1200. The I/O system 1220 can include one or more keys or a keyboard, one or more buttons, one or more displays, a speaker, a microphone, etc. that allow the user to interact with and control the computing device 1200. The I/O system 1220 also includes circuitry and a bus structure to interface with peripheral computing components such as power sources, sensors, etc.

The network interface 1225 includes transceiver circuitry that allows the computing device 1200 to transmit and receive data to/from other devices such as user device(s), remote computing systems, the 3D printer 1240, servers, websites, etc. The network interface 1225 enables communication through the network 1235, which can be one or more communication networks. The network 1235 can include a cable network, a fiber network, a cellular network, a wi-fi network, a landline telephone network, a microwave network, a satellite network, etc. The network interface 1225 also includes circuitry to allow device-to-device communication such as near field communication (NFC), Bluetooth® communication, etc.

The endoscopic sleeve application 1230 can include hardware, software, and algorithms (e.g., in the form of computer-readable instructions) which, upon activation or execution by the processor 1205, performs any of the various operations described herein such as receiving desired specifications for an endoscopic sleeve, generating a print file based on the desired specifications, controlling the 3D printer 1240 to execute the print file, controlling cleaning, controlling a curing process, controlling post-processing operations, controlling the irrigation system, controlling the light source(s), controlling the vacuum system, controlling the imaging device, etc. The endoscopic sleeve application 1230 can utilize the processor 1205 and/or the memory 1215 as discussed above.

The proposed system can be used in many applications in the ENT field. As an example, the system can be used to perform Tympanoplasty, Cholesteatoma Removal, Mastoidectomy, Stapedectomy, Cochlear Implant Surgery, Sinus Treatments, Nasal Surgeries, A denoidectomy, Laryngoscopy, Endoscopic Dacryocystorhinostomy (DCR), Salivary Gland Stone Removal, Sleep Apnea Surgery, Parotidectomy, Thyroidectomy, Parathyroidectomy, Endoscopic Ear Surgery, Vocal Cord Surgery, Airway Stenosis Treatment, Skull Base Surgery, Epistaxis Control, Sialendoscopy, Tracheal Stenosis Treatment, Ossicular Chain Reconstruction, Lateral Skull Base Surgery, Endolymphatic Sac Decompression, Rhinophyma Reduction, Septal Perforation Repair, Juvenile Angiofibroma Resection, Superior Canal Dehiscence Repair, Endoscopic Pituitary Surgery, Tonsillectomy for Chronic Tonsillitis or Sleep Apnea, Subglottic Stenosis Treatment, Endoscopic Removal of Cysts and Tumors of the Sinuses and Nasal Cavity, Orbital Decompression for Thyroid Eye Disease, Turbinate Surgery for Chronic Rhinitis, Lymph Node Biopsy for Head and Neck Cancer, Endoscopic-Assisted Repair of Choanal Atresia, Glottic and Subglottic Stenosis Treatment, Meniere's Disease Treatment, Petrous Apex Lesion Surgery, etc.

Thus, as discussed above, existing devices in the market provides illumination, suction, and irrigation separately. Conversely, the proposed system provides these functions in a single-unit, facilitating the surgery and exploratory procedures in ENT medicine, where the space is limited for navigation. Moreover, the use of a miniaturized camera and micro-LEDs instead of bundle of fibers for video acquisition and illumination, allows the possibility of bending the device in a small curvature radius, accessing hard-to-reach areas. In addition, the 3 tubing probe can be manufacture in a biocompatible clear material that aids identifying where the suction clog is located. Additionally, instead of being made from heavy, unwieldy metals, the proposed system can be made of biocompatible resin that decreases the weight of the system. As discussed herein, the proposed system enhances the efficiency and safety of surgical and exploratory procedures in a limited space in the ENT field, leading to better patient outcomes and reduced recovery times.

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

The foregoing description of illustrative embodiments of the invention has been presented for purposes of illustration and of description. It is not intended to be exhaustive or to limit the invention to the precise form disclosed, and modifications and variations are possible in light of the above teachings or may be acquired from practice of the invention. The embodiments were chosen and described in order to explain the principles of the invention and as practical applications of the invention to enable one skilled in the art to utilize the invention in various embodiments and with various modifications as suited to the particular use contemplated. It is intended that the scope of the invention be defined by the claims appended hereto and their equivalents.

What is claimed is:

1. A method of forming an endoscopic device, the method comprising:
forming, by a three-dimensional (3D) printer, a first tube of an endoscopic sleeve, wherein the first tube is sized to receive an imaging device;
forming, by the 3D printer, a second tube of the endoscopic sleeve such that at least a portion of the second tube is mounted to the first tube, wherein the second tube is sized to connect to an irrigation system; and
forming, by the 3D printer, a third tube of the endoscopic sleeve such that at least a portion of the third tube is mounted to one or more of the first tube and the second tube, wherein the third tube is sized to connect to a vacuum system.

2. The method of claim 1, wherein forming the endoscopic sleeve comprises forming the endoscopic sleeve at an orientation such that the first tube of the endoscopic sleeve is perpendicular to a ground surface upon which the 3D printer is positioned.

3. The method of claim 1, wherein the endoscopic sleeve is formed from MED-AMB 10 material, MED-WHT 10 material, or PRO-BLK 10 material.

4. The method of claim 1, wherein forming the second tube comprises forming an end portion of the second tube, wherein the end portion of the second tube is formed at a non-zero degree angle relative to the first tube such that the end portion of the second tube is not in direct contact with the first tube.

5. The method of claim 4, further comprising forming at least a portion of the end portion of the second tube as an irrigation system connector.

6. The method of claim 4, wherein forming the third tube comprises forming an end portion of the third tube, wherein the end portion of the third tube is formed at an angle relative to the first tube such that the end portion of the third tube is not in direct contact with the first tube.

7. The method of claim 6, wherein the end portion of the third tube is parallel to the end portion of the second tube.

8. The method of claim 6, further comprising forming at least a portion of the end portion of the third tube as a vacuum system connector.

9. The method of claim 6, further comprising forming, by the 3D printer, a structural support for the endoscopic sleeve, wherein the structural support includes a first portion that extends between the first tube and the end portion of the second tube.

10. The method of claim 9, wherein the first portion of the structural support is formed to be perpendicular to the first tube.

11. The method of claim 9, wherein the structural support includes a second portion that extends between the end portion of the second tube and the end portion of the third tube.

12. The method of claim 11, wherein the second portion of the structural support is formed to be parallel to the first tube.

13. The method of claim 1, wherein the first tube is sized to receive a light source to provide illumination for the imaging device.

14. The method of claim 1, further comprising forming a distal end of the second tube such that the distal end is angled toward a distal end of the first tube such that the irrigation system is able to clean a lens of the imaging device.

15. The method of claim 1, further comprising cleaning an interior and an exterior of the endoscopic sleeve with alcohol.

16. An endoscopic system comprising:
a first tube of an endoscopic sleeve, wherein the first tube is sized to receive an imaging device;
a second tube of the endoscopic sleeve, wherein the second tube is sized to connect to an irrigation system; and
a third tube of the endoscopic sleeve, wherein the third tube is sized to connect to a vacuum system; and
a controller in communication with the vacuum system and the irrigation system, wherein the controller controls the vacuum system and the irrigation system.

17. The system of claim 16, wherein the second tube includes an end portion that is formed at a non-zero degree angle relative to the first tube such that the end portion of the second tube is not in direct contact with the first tube.

18. The system of claim 17, wherein the third tube includes an end portion that is formed at an angle relative to the first tube such that the end portion of the third tube is not in direct contact with the first tube and such that the end portion of the third tube is parallel to the end portion of the second tube.

19. The system of claim 18, further comprising a structural support for the endoscopic sleeve, wherein the structural support includes a first portion that extends between the first tube and the end portion of the second tube.

20. The system of claim 19, further comprising a second portion of the structural support that extends between the end portion of the second tube and the end portion of the third tube.
